(19) United States
(12) Patent Application Publication
(10) Pub. No.: US 2004/0175716 A1
(43) Pub. Date: Sep. 9, 2004

(54) Dna sequence analysis

(76) Inventors: Shankar Balasubramanian, Cambridge (GB); David Klenerman, Cambridge (GB); Colin Barnes, Nr Saffron Walden (GB); Alan Rowe Williamson, Beaconsfield (GB)

Correspondence Address: KLAUBER & JACKSON, 411 HACKENSACK AVENUE, HACKENSACK, NJ, 07601

(21) Appl. No.: 10/486,951

(22) PCT Filed: Aug. 13, 2002
(86) PCT No.: PCT/GB02/03750

(30) Foreign Application Priority Data
Aug. 13, 2001 (GB) 0119719.3

(51) Int. Cl. C12Q 1/68; C12P 19/34

(52) U.S. Cl. 435/6; 435/91.2

(57) ABSTRACT

The present invention concerns a method for determining the identity of one or more single nucleotide polymorphisms (SNP) in a genome, comprising: (i) fragmenting a sample genome; (ii) contacting the fragments with an excess of a plurality of different oligonucleotide primers under conditions that permit a primer to form a duplex with a complementary region on a fragment, each primer having a predetermined sequence complementary to a sequence on the genome that is proximal to a putative SNP site, and the resulting duplexes being immobilised on a solid support; (iii) carrying out the sequencing reaction(s) and detecting the incorporation of bases onto the oligonucleotide primers to extend the primers to at least the SNP site; and (iv) comparing the resulting sequences to those of the reference one or more SNPs.

### FIELD OF THE INVENTION

This invention relates to a method for detecting variations in the sequences of nucleic acid fragments, particularly in the DNA sequences of genes in a sample obtained from a patient.

### BACKGROUND OF THE INVENTION

Recently, the Human Genome Project determined the entire sequence of the human genome—all 3×10⁹ bases. The sequence information represents that of an average human. However, there is still considerable interest in identifying differences in the genetic sequence between different individuals. The most common form of genetic variation is single nucleotide polymorphisms (SNPs). On average one base in 1000 is a SNP, which means that there are 3 million SNPs for any individual. Some of the SNPs are in coding regions and produce proteins with different binding affinities or properties. Some are in regulatory regions and result in a different response to changes in levels of metabolites or messengers. SNPs are also found in non-coding regions, and these are also important as they may correlate with SNPs in coding or regulatory regions. The key problem is to develop a low cost way of determining one or more of the SNPs for an individual.

Nucleic acid arrays have been used to determine SNPs, usually in the context of monitoring hybridisation events (Mirzabekov, Trends in Biotechnology (1994) 12:27-32). Many of these hybridisation events are detected using fluorescent labels attached to nucleotides, the labels being detected using a sensitive fluorescent detector, e.g. a charge-coupled detector (CCD). The major disadvantage of these methods is that repeat sequences can lead to ambiguity in the results. This problem is recognised in Automation Technologies for Genome Characterisation, Wiley-Interscience (1997), ed. T. J. Beugelsdijk, Chapter 10: 205-225.

Other analysis methods require the sequencing of genomic fragments using high-density polynucleotide arrays. The use of high-density arrays in a multi-step analysis procedure can lead to problems with phasing. Phasing problems result from a loss in the synchronisation of a reaction step occurring on different molecules of the array. If some of the arrayed molecules fail to undergo a step in the procedure, subsequent results obtained for these molecules will no longer be in step with results obtained for the other arrayed molecules. The proportion of molecules out of phase will increase through successive steps and consequently the results detected will become ambiguous. This problem is recognised in the sequencing procedure described in U.S. Pat. No. 5,302,509.

An alternative sequencing approach is disclosed in EP-A-0381693, which comprises hybridising a fluorescently-labelled strand of DNA to a target DNA sample suspended in a flowing sample stream, and then using an exonuclease to cleave repeatedly the end base from the hybridised DNA. The cleaved bases are detected in sequential passage through a detector, allowing reconstruction of the base sequence of the DNA. Each of the different nucleotides has a distinct fluorescent label attached, which is detected by laser-induced fluorescence. This is a complex method, primarily because it is difficult to ensure that every nucleotide of the DNA strand is labelled and that this has been achieved with high fidelity to the original sequence.

### SUMMARY OF THE INVENTION

The present invention is based on the realisation that the information provided by sequencing projects such as the Human Genome Sequencing Project can be used to design specific primer sequences that can be used to hybridise to regions near a SNP site on a sample genome (or genomic fragment), to provide a starting point for a limited sequence determination to be made. The base incorporated at the SNP site can then be compared with a reference sequence to determine whether it is the same as the reference sequence. Multiple primers can be used in one experiment. This obviates the need to sequence the entire genome to identify multiple SNP sites, leading to a reduction in costs and processing time.

Therefore, according to the invention, there is provided a method for determining the identity of one or more single nucleotide polymorphisms (SNP) in a genome, comprising:

(i) fragmenting a sample genome;

(ii) contacting the fragments with an excess of a plurality of different oligonucleotide primers under conditions that permit a primer to form a duplex with a complementary region on a fragment, each primer having a predetermined sequence complementary to a sequence on the genome that is proximal to a putative SNP site, and the resulting duplexes being immobilised on a solid support;

(iii) carrying out the sequencing reaction(s) and detecting the incorporation of bases onto the oligonucleotide primers to extend the primers to at least the SNP site; and

(iv) comparing the resulting sequences to those of the reference SNPs.

### DESCRIPTION OF THE INVENTION

The present invention relates to a method that can be used to sequence short fragments of a sample genome, to identify the sequences of multiple SNPs. The present invention is therefore useful to determine whether a subject has a particular SNP, and therefore a risk of disease. Many cancers are caused by genetic mutation on particular genes, for example a single mutation is implicated in breast cancer. The methods of the present invention can be used to screen for a wide variety of mutations that have been shown to be implicated in disease. The ability to screen for multiple (e.g. thousands) potential SNPs in a single experiment is therefore of great benefit.

The method relies on the ability to utilise the information provided by genome sequencing efforts, such as the Human Genome Project, to compare short sequences in a sample with a reference or wild-type sequence, to identify any aberrations. SNP sites are known, and it is possible to use this information to design oligonucleotide primers that are complementary to sequences on the genome close to (e.g. adjacent) the SNP site. By hybridising a plurality of primers to fragments of a sample genome close to SNP sites, only limited sequencing is required to gain information on each SNP site. Using the limited sequence information generated, and knowledge of the reference or wild-type sequence, it is possible to identify the location of each sequenced fragment on the genome, and to identify the sequence of the SNP present.

The method is to be carried out so that the base incorporation can be determined for individual duplexes. In the preferred method, single molecule imaging is used to monitor the incorporation of bases onto each primer at the single molecule level. Further details of single molecule imaging are given below, and are also disclosed in international patent publication no. WO-A-00/06770, the content of which is hereby incorporated by reference.

The oligonucleotide primers may comprise from 10 to 70 bases, preferably 15 to 60 bases, more preferably 30 to 50 bases, and most preferably about 40 bases. As a mixture of primers are to be used, it is possible to use primers of different lengths in the one reaction. If a mixture of different length primers are used, the average length of the primers is specified above. It is preferable to adjust the number of bases on each primer to normalise the melting temperature and thus ensure efficient hybridisation of each primer under the universal hybridisation conditions. It is preferable to design each primer so that it is complementary to a sequence less than 20 bases from the SNP site, more preferably less than 10 bases, and most preferably from 1 to 6 bases. The primer may be adjacent to the SNP site.

The number of bases that need to be sequenced will be determined by the position of the SNP site, and the number of different primers used. The more primers added, the more bases that may need to be sequenced in order to identify which primer is associated with the genomic fragment and which SNP is being determined.

For example, if there are 1000 different primers used, it will usually be necessary to determine the incorporation of at least 5 bases, to accurately identify the primer used. The SNP site will be located at a known position within the sequenced bases. If 10,000 different primers are to be used, it will usually be necessary to sequence 7 bases to accurately determine each primer. Any number of different primers can be used, provided that the detection of base incorporation is carried out in a way that distinguishes the different primers. In the context of single molecule imaging, it is preferable to have from 300 to 10⁶ different primers, more preferably 10³ to 10⁴ different primers. Smaller numbers of different primers, e.g. 300 to 1000, preferably 400 to 600 different primers may be used if it is desired to restrict the analysis to a small number of defined SNP sites. The primers are present in excess compared to the concentration of genomic fragments.

The sample genomic DNA may be obtained by methods known in the art. Fragmentation may be carried out by any suitable method, including restriction enzyme digestion and the use of shear forces.

The primers are preferably brought into contact with the fragments in solution under hybridising conditions, so that duplex formation occurs between complementary primer sequences and genomic fragments. Hybridising conditions are known in the art and suitable buffers, salt concentrations, temperatures etc will all be apparent to the skilled person. After the hybridisation step, the resulting duplexes are immobilised onto a solid support.

Immobilisation of the duplexes to the surface of a solid support may be carried out by techniques lcnown in the art to form an array, which in one embodiment, as set out in more detail below, may provide adequate separation for individual resolution of the duplexes. In the context of the present invention, an array refers to a population of polynucleotide molecules distributed over the solid support. Generally the array is produced by dispensing small volumes of a sample to generate a random single molecule array. In this manner, a mixture of different molecules may be arrayed by simple means to produce a single molecule array. In this embodiment, both duplexed and non-duplexed fragments will be immobilised onto the solid support. However, those fragments that are not duplexed will not undergo the sequencing reaction and so will not generate a detectable signal. It is also possible, in an alternative embodiment, to design the primers so that they incorporate a chemical moiety prior to hybridisation that permits attachment to the solid surface.

In a preferred embodiment of the invention duplexed molecules are attached to the solid support via covalent linkage to the genomic fragment, which is preferably carried out prior to hybridisation. This may be achieved by various techniques including, preferably, the incorporation of a nucleotide onto one end of the fragment, the nucleotide being modified with a linker molecule that reacts with a suitably prepared solid support. The modified nucleotide can be incorporated onto the genomic fragment in a conventional way using a terminal transferase or polymerase. This incorporation step may be carried out prior to the hybridisation step with the oligonucleotide primer. It is also possible to immobilise the genomic fragments to the solid support prior to the addition of the primers. However, it is more preferable to carry out the hybridisation step in solution and then immobilise, as this is more flexible in terms of the concentrations of fragments and primers that can be used in the hybridisation step.

It is also possible to immobilise the primers to the solid support, prior to hybridisation with the genomic fragments. The primers may be immobilised on a solid support either randomly or non-randomly. If the primers are immobilised non-randomly, it is possible to design all the primers so that the SNP site is adjacent the primer, thereby requiring only the incorporation of one base to characterise the SNP site.

On formation of the duplex, it may be preferable to attach the primer to the genomic fragment by a chemical linkage. This may be done using known cross-linking reagents, including the use of sulphydryl groups.

Solid supports that are suitable for use in the invention are available commercially, and will be apparent to the skilled person. The supports may be manufactured from materials such as glass, ceramics, silica and silicon. The supports usually comprise a flat (planar) surface. Any suitable size may be used. For example, the supports might be of the order of 1 to 10 cm in each direction.

Immobilisation may be by specific covalent or non-covalent interactions. Covalent attachment is preferred. However, the polynucleotide can be attached to the solid support at any position along its length, the attachment acting to tether the polynucleotide to the solid support. The immobilised polynucleotide is then able to undergo interactions at positions distant from the solid support. Typically the interaction will be such that it is possible to remove any molecules bound to the solid support through non-specific interactions, e.g. by washing. Immobilisation in this manner results in well separated single polynucleotides.

In a preferred embodiment of the invention, the solid surface is coated with an epoxide and the duplexed molecules are coupled to the support via an amine linkage. It is also preferable to avoid or reduce salt present in the solution containing the molecule to be arrayed. Reducing the salt concentration minimises the possibility of the molecules aggregating in the solution, which may affect the positioning on the array.

After immobilisation, the incorporation of bases onto the primers (i.e. complementary to the genomic fragment) can be determined, and this information used to identify SNP present. Conventional assays which rely on the detection of fluorescent labels attached to the bases can be used to obtain the information on the SNP. These assays rely on the stepwise identification of suitably labelled bases, referred to in U.S. Pat. No. 5,634,413 as “single base” sequencing methods. The bases are incorporated onto the primer sequence using the polymerase reaction.

In an embodiment of the invention, the incorporation of bases is determined in a similar manner to that described in U.S. Pat. No. 5,634,413, using fluorescently labelled nucleotides. The nascent chain (on the primer) is extended in a stepwise manner by the polymerase reaction. Each of the different nucleotides (A, T, G and C) incorporates a unique fluorophore at the 3′ position which acts as a blocking group to prevent uncontrolled polymerisation. As used herein, the term “blocking group” refers to a moiety attached to a nucleotide which, while not interfering substantially with template-dependent enzymatic incorporation of the nucleotide into a polynucleotide chain, abrogates the ability of the incorporated nucleotide to serve as a substrate for further nucleotide addition. A “removable blocking group” is a blocking group that can be removed by a specific treatment that results in the cleavage of the covalent bond between the nucleotide and the blocking group. Specific treatments can be, for example, a photochemical, chemical or enzymatic treatment that results in the cleavage of the covalent bond between the nucleotide and the fluorescent label. Removal of the blocking group will restore the ability of the incorporated, formerly blocked nucleotide to serve as a substrate for further enzymatic nucleotide additions. The polymerase enzyme incorporates a nucleotide into the nascent chain complementary to the sequence on the genomic fragment, and the blocking group prevents further incorporation of nucleotides. Unincorporated nucleotides are removed and each incorporated nucleotide is “read” optically by a charge-coupled detector using laser excitation and filters. The 3′-blocking group is then removed (deprotected), to expose the nascent chain for further nucleotide incorporation.

Because the array consists of distinct optically resolvable polynucleotides, each target polynucleotide will generate a series of distinct signals as the fluorescent events are detected. Details of the sequence are then determined and can be compared with known sequence information to identify SNPs.

The number of cycles that can be achieved is governed principally by the yield of the deprotection cycle. If deprotection fails in one cycle, it is possible that later deprotection and continued incorporation of nucleotides can be detected during the next cycle. Because the sequencing is performed at the single molecule level, the sequencing can be carried out on different polynucleotide sequences at one time without the necessity for separation of the different sample fragments prior to sequencing. This sequencing also avoids the phasing problems associated with prior art methods.

The labelled nucleotides can comprise a separate label and removable blocking group, as will be appreciated by those skilled in the art. In this context, it will usually be necessary to remove both the blocking group and the label prior to firther incorporation.

Deprotection can be carried out by chemical, photochemical or enzymatic reactions. A similar, and equally applicable, sequencing method is disclosed in EP-A-0640146. Other suitable sequencing procedures will be apparent to the skilled person.

The images and other information about the arrays, e.g. positional information, etc. are processed by a computer program which can perform image processing to reduce noise and increase signal or contrast, as is known in the art. The computer program can perform an optional alignment between images and/or cycles, extract the single molecule data from the images, correlate the data between images and cycles and specify the DNA sequence from the patterns of signal produced from the individual molecules.

In a preferred embodiment of the invention, the duplex is immobilised on a solid support surface at a density that allows each duplex to be individually resolved by optical means, i.e. single molecule imaging. This means that, within the resolvable area of the particular imaging device used, there must be one or more distinct images each representing one duplex. Typically, the detection of incorporated bases can be carried out using a single molecule fluorescence microscope equipped with a sensitive detector, e.g. a charge-coupled detector (CCD). Each duplex of the array may be analysed simultaneously or, by scanning the array, a fast sequential analysis can be performed. Methods for the preparation of single molecule arrays and for single molecule imaging are described in WO-A-00/06770.

The term “individually resolved” is used herein to indicate that, when visualised, it is possible to distinguish one duplex on the array from neighbouring duplexes. Visualisation may be effected by the use of the detectably-labelled nucleotides as discussed above.

The density of the arrays is not critical. However, the present invention can make use of a high density of immobilised molecules, and these are preferable. For example, arrays with a density of 10⁶to 10⁹ and preferably 10⁸ duplexed molecules per cm² may be used. Preferably, the density is at least 10⁷/cm² and typically up to 10⁸/cm². These high density arrays are in contrast to other arrays which may be described in the art as “high density” but which are not necessarily as high and/or which do not allow single molecule resolution. On a given array, it is the number of single polynucleotides, rather than the number of features, that is important. The concentration of nucleic acid molecules applied to the support can be adjusted in order to achieve the highest density of addressable single polynucleotide molecules. At lower application concentrations, the resulting array will have a high proportion of addressable single polynucleotide molecules at a relatively low density per unit area. As the concentration of nucleic acid molecules is increased, the density of addressable single polynucleotide molecules will increase, but the proportion of single polynucleotide molecules capable of being addressed will actually decrease. One skilled in the art will therefore recognize that the highest density of addressable single polynucleotide molecules can be achieved on an array with a lower proportion or percentage of single polynucleotide molecules relative to an array with a high proportion of single polynucleotide molecules but a lower physical density of those molecules.

Using the methods and apparatus of the present invention, it may be possible to image at least 10⁷ or 10⁸ molecules simultaneously. Fast sequential imaging may be achieved using a scanning apparatus; shifting and transfer between images may allow higher numbers of duplexed molecules to be imaged.

The extent of separation between the individual duplexed molecules on the array will be determined, in part, by the particular technique used for resolution. Apparatus used to image molecular arrays are known to those skilled in the art. For example, a confocal scanning microscope may be used to scan the surface of the array with a laser to image directly a fluorophore incorporated on the individual molecule by fluorescence. Alternatively, a sensitive 2-D detector, such as a charge-coupled detector, can be used to provide a 2-D image representing the individual duplexed molecules on the array.

Resolving single molecules on the array with a 2-D detector can be done if, at 100× magnification, adjacent duplexed molecules are separated by a distance of approximately at least 250 nm, preferably at least 300 nm and more preferably at least 350 nm. It will be appreciated that these distances are dependent on magnification, and that other values can be determined accordingly, by one of ordinary skill in the art.

Other techniques such as scanning near-field optical microscopy (SNOM) are available which are capable of greater optical resolution, thereby permitting more dense arrays to be used. For example, using SNOM, adjacent duplexed molecules may be separated by a distance of less than 100 nm, e.g. 10 nm. For a description of scanning near-field optical microscopy, see Moyer et al., Laser Focus World (1993) 29(10).

An additional technique that may be used is surface-specific total internal reflection fluorescence microscopy (TIRFM); see, for example, Vale et al., Nature, (1996) 380: 451-453). Using this technique, it is possible to achieve wide-field imaging (up to 100 μm×100 μm) with single molecule sensitivity. This may allow arrays of greater than 10⁷ resolvable molecules per cm² to be used.

Additionally, the techniques of scanning tunnelling microscopy (Binnig et al., Helvetica Physica Acta (1982) 55:726-735) and atomic force microscopy (Hansma et al., Ann. Rev. Biophys. Biomol. Struct. (1994) 23:115-139) are suitable for imaging the arrays of the present invention. Other devices which do not rely on microscopy may also be used, provided that they are capable of imaging within discrete areas on a solid support.

The sequence information obtained from the polymerase reaction can be compared to a reference sequence to identify the SNPs. The reference sequence is any suitable sequence that represents the normal/general genome. Suitable reference genomes have been identified as part of the various genome sequencing efforts, for example the Human Genome Project. It is, strictly, only the base at the SNP site that is compared with the corresponding base on the reference sequence. The remaining sequence (primer and additional sequenced bases) is used to identify the relevant part of the reference sequence under study.

1. A method for determining the identity of one or more single nucleotide polymorphisms (SNP) in a genome, comprising:
(i) fragmenting a sample genome;
(ii) contacting the fragments with an excess of a plurality of different oligonucleotide primers under conditions that permit a primer to form a duplex with a complementary region on a fragment, the primers having a predetermined sequence complementary to a sequence on the genome that is proximal to a SNP site, and the resulting duplexes being immobilised on a solid support;
(iii) carrying out the sequencing reaction(s) and detecting the incorporation of bases onto the oligonucleotide primers to extend the primers to at least the SNP site; and
(iv) comparing the resulting bases to those of the reference one or more SNPs.

2. A method according to claim 1, wherein the duplex is immobilised to the solid support via a covalent linkage to the fragment.

3. A method according to claim 1 or claim 2, wherein prior to step (ii), a nucleotide is incorporated onto one end of the fragments, the nucleotide comprising a linker molecule for immobilisation of the fragments with the solid support.

4. A method according to any of claims 1 to 3 wherein immobilisation is at a density that allows each immobilised duplex to be individually resolved by optical microscopy.

5. A method according to any preceding claim, wherein step (ii) comprises between 300 to 10⁶ different oligonucleotide primers.

6. A method according to any preceding claim, wherein step (ii) comprises from 10³ to 10⁵ different oligonucleotide primers.

7. A method according to any preceding claim, wherein step (ii) comprises from 10³ to 10⁴ different oligonucleotide primers.

8. A method according to any preceding claim, wherein the oligonucleotide primers comprise from 10 to 70 bases.

9. A method according to any preceding claim, wherein the oligonucleotide primers comprise from 30 to 50 bases.

10. A method according to any preceding claim, wherein the oligonucleotide primers comprise about 40 bases.

11. A method according to any preceding claim, wherein the primers are complementary to a sequence less than 20 bases from the SNP site.

12. A method according to any preceding claim, wherein the primers are complementary to a sequence less than 10 bases from the SNP site.

13. A method according to any preceding claim, wherein the primers are complementary to a sequence from 1 to 6 bases from the SNP site.

14. A method according to any preceding claim, wherein the primers are complementary to a sequence adjacent to the SNP site.

15. A method according to any preceding claim, wherein step (iii) comprises the sequential addition of fluorescently-labelled bases.
